(19) United States
(12) Patent Application Publication
(10) Pub. No.: US 2025/0248687 A1
(43) Pub. Date: Aug. 7, 2025

(54) MECHANICAL FOUR-DIMENSIONAL INTRACARDIAC ULTRASOUND IMAGING SYSTEM

(71) Applicant: SHENZHEN CARDIOACC LTD, Shenzhen (CN)

(72) Inventors: Xinhuan ZHOU, Shenzhen (CN); Jun MA, Shenzhen (CN); Jiahui ZHAO, Shenzhen (CN); Yuqin LIU, Shenzhen (CN)

(73) Assignee: SHENZHEN CARDIOACC LTD, Shenzhen (CN)

(21) Appl. No.: 19/186,735

(22) Filed: Apr. 23, 2025

Related U.S. Application Data
(63) Continuation of application No. PCT/CN2023/135824, filed on Dec. 1, 2023.

(30) Foreign Application Priority Data
Nov. 6, 2023 (CN) 202311466878.X

(51) Int. Cl. A61B 8/00; A61B 8/08; A61B 8/12; A61B 50/20

(52) U.S. Cl. CPC A61B 8/4461; A61B 8/0883; A61B 8/12; A61B 8/445; A61B 8/4488; A61B 8/461; A61B 8/5215; A61B 50/20

(57) ABSTRACT

A rotating four-dimensional intracardiac ultrasound imaging system including an ultrasound system, a slip ring, a host end connector, a catheter end connector, a motor, a one-dimensional transducer, a sheath and a torque coil is introduced. The ultrasound system is connected to the transducer through the slip ring, two connectors, a cable and the torque coil in sequence. The motor drives the slip ring, connectors, torque coil and transducer to rotate unidirectionally and uniformly at a high speed. Meanwhile, the transducer is for two-dimensional imaging and acquires multiple two-dimensional images uniformly around the rotation axis. Then multiple two-dimensional images acquired at different rotation angles are reconstructed as four-dimensional images. Compared with existing four-dimensional intracardiac ultrasound using a complicated and expensive transducer, the present disclosure significantly reduces the cost of the transducer and the catheter, and has a larger imaging field of view (up to) 360° in a rotation direction.

### CROSS-REFERENCE TO RELATED APPLICATIONS

This application is a Bypass Continuation of the PCT application with the international application number PCT/CN2023/135824 filed on Dec. 1, 2023, designating the United States, now pending, and further claims foreign priority to Chinese Patent Application No. 202311466878.X filed on Nov. 6, 2023.

### TECHNICAL FIELD

The present disclosure relates to the field of ultrasound imaging, in particular to a mechanical four-dimensional intracardiac ultrasound imaging system.

### BACKGROUND

Heart disease is an important human health problem, including valve disease (such as valvular stenosis and dysregulation), arrhythmia (such as atrial fibrillation and atrial flutter), myocardial hypertrophy, etc. Catheter intervention is the first-line clinical treatment means of cardiac diseases due to minimal invasive surgery and safety. Accurate four-dimensional imaging of cardiac structure is a precondition for safe and effective operation in various cardiac interventional surgeries. For example, in the process of intracardiac 3D mapping and ablation, four-dimensional imaging is needed to determine the contact of the catheter and the endocardium and the relative position of the focal target. Four-dimensional imaging technology can provide real-time 3D image guidance for intracardiac interventional operations, thereby reducing the difficulty of operations and improving the safety of operations, as well as reducing X-ray radiation.

The transducers for four-dimensional intracardiac ultrasound usually use a complex and expensive two-dimensional matrix sensor integrated with an ASIC (Application Specific Integrated Circuit) chip. The intracardiac ultrasound catheter is disposable, so the cost is high. It is of great clinical and economic values to develop a new technical route to reduce the cost of existing four-dimensional intracardiac ultrasound imaging transducer.

### SUMMARY

In view of the above problem, the embodiment of the present disclosure is proposed to provide a mechanical four-dimensional intracardiac ultrasound imaging system overcoming the above problem or at least partially solving the above problem.

The embodiment of the present disclosure provides a mechanical four-dimensional intracardiac ultrasound imaging system. The system at least includes an ultrasound system, a slip ring, a host end connector, a catheter end connector, a motor, a one-dimensional phase array transducer, a sheath and a torque coil, wherein the ultrasound system is connected to the host end connector via the slip ring, and the catheter end connector is connected to the transducer via a cable and the torque coil.

The motor is configured to drive the slip ring to drive the two connectors to rotate uniformly and unidirectionally at a high speed.

The slip ring includes a rotor side and a stator side, wherein the stator side is directly connected with the host end connector, and the rotor side is connected with the catheter end connector. The motor drives the rotor side of the slip ring to rotate, thereby driving the two connectors, the torque coil and the transducer to rotate around the center of the transducer in the sheath.

The torque coil is configured to drive the ultrasound transducer to rotate within the sheath.

The ultrasound transducer is a one-dimensional phase array transducer and configured to transmit and receive ultrasound beams in different space directions.

The ultrasound system is configured to transmit ultrasound beams in different directions. The ultrasound transducer may be side-looking (FIG. 1) or forward-looking (FIG. 2). The two-dimensional images formed at different rotation angles are then reconstructed to form three-dimensional images, followed by rendering and real-time display of reconstructed four-dimensional images. For the instance (FIG. 1) where the transducer is side-looking, a four-dimensional image is generated every time the transducer rotates 360°. For the instance (FIG. 2) where the transducer is forward-looking, a two-dimensional image rotating at a high speed around the central axis is generated, and a four-dimensional image is generated every time the transducer rotates 180°.

Optionally, the slip ring includes a stator side and a rotor side. The rotor side is connected with the rotating motor and rotates, and the stator side is connected to the ultrasound system and static. The rotor side and the stator side move relatively and are electrically connected via an electric brush structure.

Optionally, the transducer is a one-dimensional phase array transducer and rotates unidirectionally and uniformly at a high speed around the center as a rotation center.

Optionally, a water inlet with a one-way valve is formed in a proximal end of the catheter, and an air outlet is formed in a distal end of the catheter. The distal end of the catheter is made of silica gel. A coupling fluid (such as normal saline) is filled in the water inlet in the proximal end of the catheter, and air is exhausted through the air outlet.

Optionally, the ultrasound system transmits ultrasound beams in different directions and receives the reflected echoes, meanwhile the transducer rotates around the center to generate multiple two-dimensional images at different rotation angles. Then these two-dimensional images are quickly three-dimensionally reconstructed, followed by being rendered and displayed in the monitor. For side-looking and forward-looking schemes, the rotation centers are different (see FIG. 1 and FIG. 2). The technology includes:
obtaining the coordinate position of each voxel on the ultrasound beam and the distance to each channel of the ultrasound transducer, and calculating transmit and receive delays;

Conducting ultrasound beamforming to reconstruct the two-dimensional images, then calculating the space angle of each voxel point of the 2D image in spherical coordinates according to each image's corresponding rotation angle. The transducer rotates uniformly and thus the rotation angle is the multiplication of rotation speed and time;
after the ultrasound transducer rotates around the central axis by 180° (forward looking) or 360° (side looking) each time, performing fast three-dimensional reconstruction on all the two-dimensional images in a Cartesian coordinate system according to the space angle, wherein therefore, when the four-dimensional volumetric frame rate is set to f, the rotational speed of the motor, the slip ring and the transducer is 0.5*f (forward looking) or f(side looking);
performing three-dimensional interpolation from the spherical coordinates to Cartesian coordinates, according to the space angle and the beamformed two-dimensional images;
performing rendering and image display of the generated four-dimensional images.

Optionally, when the transducer unidirectionally and uniformly rotates at a high speed, the transducer transmits focused ultrasound beams in sequence to scan different angles in a two-dimensional plane, or transmits a sequence of non-focused plane waves or divergent waves in sequence in a two-dimensional plane, and receives reflected echo signals.

Optionally, the transducer is connected with the catheter end connector through a wire harness, the transducer wire harness being a coaxial cable or a flexible circuit board. The transducer, the wire harness and the catheter end connector rotate uniformly inside the sheath, and the sheath remains static.

Optionally, the transducer wire harness, the transducer and the torque coil are located in the sheath. When in use, a doctor injects coupling fluid into the proximal end of the catheter and air is exhausted out of the ultrasound transducer and the sheath to form acoustic coupling between the transducer and the sheath.

Optionally, the sheath is a deflectable sheath with gradually changing hardness. The catheter has a relatively soft distal end and a relatively hard proximal end. Pull wires are integrated in the catheter wall of the catheter. The catheter is deflected by pulling the pull wires through operation of a handle.

Optionally, the motor and the slip ring are integrated in a driver. The driver is capable of being put on a hospital bed or located on an adjustable support arm, and the distance from the catheter to a patient can be adjusted by moving the driver.

The embodiment of the present disclosure has the following advantages.

The embodiment of the present disclosure provides a mechanical four-dimensional intracardiac ultrasound imaging system. The system includes an ultrasound system, a slip ring, a host end connector, a catheter end connector, a rotating motor, a transducer, cables, an ultrasound transducer sheath and a torque coil. Wherein, the ultrasound system is connected to the transducer through the slip ring, the connectors, a cable and the torque coil in sequence. When in use, the motor drives the multi-channel slip ring to rotate unidirectionally, thereby driving the host end connector, the catheter end connector, the torque coil, the ultrasound transducer and the cable inside the sheath to rotate unidirectionally and uniformly at a high speed. At the same time, the transducer is configured to transmit ultrasound beams in different directions and receives the reflected echoes. The ultrasound system is configured to generate multiple frames of two-dimensional images around the central axis with the rotating transducer, and the two-dimensional images formed at different rotation angles are quickly three-dimensionally reconstructed to obtain four-dimensional images for final rendering and real-time display. Compared with an existing four-dimensional intracardiac ultrasound which uses a two-dimensional array transducer with integrated ASIC chip, the mechanical four-dimensional intracardiac ultrasound in the present disclosure can obviously reduce the complexity of transducer and thus the cost of the catheter.

### BRIEF DESCRIPTION OF THE FIGURES

The attached figures are used in order to illustrate the present disclosure more clearly. Apparently, the embodiments in the following description are merely a part rather than all embodiments of the present disclosure. For those of ordinary skill in the art, under the premise of without contributing creative labor, other attached figures further can be obtained according to these attached figures.

Wherein,

FIG. 1 is a structure diagram of a side-looking mechanical four-dimensional intracardiac ultrasound imaging system in an embodiment of the present disclosure.

FIG. 2 is a structure diagram of a forward-looking mechanical four-dimensional intracardiac ultrasound imaging system in an embodiment of the present disclosure.

FIG. 3 is a schematic diagram of different ultrasound transducers in the present disclosure.

FIG. 4 is a schematic diagram of a two-dimensional imaging scanning method in the absence of rotation in the present disclosure.

FIG. 5 is a schematic diagram of a three-dimensional imaging scanning method of a forward-looking mechanical four-dimensional intracardiac ultrasound imaging system in the present disclosure, with transducer rotating.

FIG. 6 is a schematic diagram of conversion between spherical coordinate and Cartesian coordinate.

### DETAILED DESCRIPTION OF THE EMBODIMENTS

Embodiments in the present disclosure are described in detail below. The embodiments described below are exemplary and are only used for explaining the present disclosure and are not construed as limiting the present disclosure.

It needs to be noted that, the terms “first” and “second” are merely intended for a purpose of description, and shall not be understood as an indication or implication of relative importance. Therefore, a feature limited by “first” or “second” may explicitly or implicitly include one or more features. Further, in the descriptions of the embodiments of the present invention, “plurality” means at least two, unless otherwise specified.

In this paper, the terms “contain” or “include” is an open expression, including content indicated by the present disclosure, but not excluding content in other aspects, so that a process, a method, an article, or a device that includes a list of elements not only includes those elements but also includes other elements that are not expressly listed, or further includes elements inherent to such a process, method, article, or device. In absence of more constraints, an element preceded by “includes a” does not preclude the existence of other identical elements in the process, method, article, or electronic equipment that includes the element.

In this paper, the terms “optionally”, “selectively” or “optional” generally mean that the event or situation described later may or may not occur, and the description includes the situation in which the event or situation occurs and the situation in which the event or situation does not occur.

The present disclosure provides a mechanical four-dimensional intracardiac ultrasound imaging system. The system will be described in detail below.

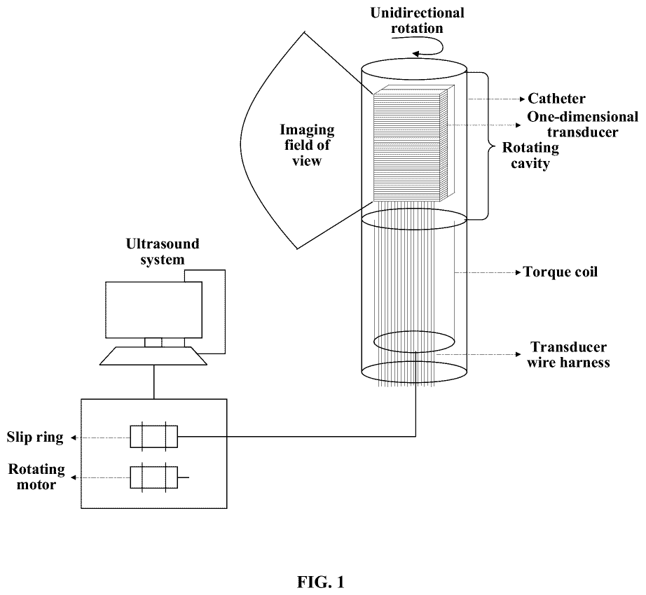

FIG. 1 shows a structure diagram of a mechanical four-dimensional intracardiac ultrasound imaging system in an embodiment of the present disclosure. The system at least includes:

an ultrasound system, a slip ring, a host end connector, a catheter end connector, a motor, a transducer, a sheath and a torque coil, wherein the ultrasound system is connected to the host end connector through the slip ring, and the catheter end connector is connected to the transducer through a cable and the torque coil.

The motor is configured to drive a rotor side of the slip ring, the system end connector and the catheter end connector to rotate uniformly at a high speed.

The slip ring includes a rotor side and a stator side, wherein the stator side is directly connected with the host end connector, and wherein the rotor side is connected with a catheter. The motor drives the rotor side of the slip ring to rotate, as well as the torque coil and the transducer to rotate around the center of the transducer in the sheath. The rotor side and the stator side of the slip ring are connected electrically via brush.

The torque coil is configured to drive the ultrasound transducer to rotate within the sheath, and the sheath remains static.

The ultrasound transducer is configured to transmit ultrasound beams in different directions and receive the reflected echoes. The transmitted beam can be focused beam or non-focused beams including divergent wave or plane wave.

The ultrasound system is configured to generate two-dimensional images rotating around a central axis at a high speed with the motor and transducer rotating, and the two-dimensional images formed at different rotation angles are three-dimensionally reconstructed followed by rendering and real-time display on monitors.

According to a novel side-looking or forward-looking mechanical rotating four-dimensional intracardiac echocardiography imaging technology provided by an embodiment of the present disclosure, a one-dimensional phase array ultrasound transducer is used. The motor and slip ring are connected to the ultrasound system. When the motor rotates, the host end connector, the catheter end connector, the torque coil, the cables and the transducer are driven to rotate to generate four-dimensional scanning. Therefore, a simple and chip one-dimensional phase array transducer can be used for four-dimensional imaging, instead of using a complicated and expensive two-dimensional phase array transducer in existing four-dimensional intracardiac echocardiography products. Forward-looking or side-looking imaging is adopted in the embodiment of the present disclosure. Particularly, when forward-looking imaging is used, two three-dimensional images are acquired every time the motor and the slip ring rotate by 360°. So for forward-looking system, the imaging frame rate is twice higher than the side looking system. For example, the rotational speed of the motor and the slip ring needs to be 10 rps for a four-dimensional imaging frame rate of 20 vol/s for forward-looking system, while the rotational speed has to be 20 rps for a four-dimensional imaging frame rate of 20 vol/s for side-looking system.

Optionally, the slip ring includes a stator side and a rotor side. The rotor side is connected with the rotating motor and rotates, and the stator side is connected to the ultrasound system and static. The rotor side and the stator side move relatively and are electrically connected via an electric brush.

Optionally, the transducer is a one-dimensional phase array transducer and rotates unidirectionally and uniformly at a high speed around the rotation center.

Optionally, the transducer at least includes configuration parameters in a scheme including center frequency, bandwidth, number of elements, pitch and element arrangement.

Specifically, various parameters of a transducer array are not limited, and arrays in different schemes including center frequency, bandwidth, number of elements, pitch and element arrangement can be adjusted according to various applications.

Optionally, in the side-looking imaging scheme, a three-dimensional image can be obtained every time the transducer rotates by 360° around the center. When the volumetric frame rate of four-dimensional ultrasound imaging is f, the rotational speed of the motor and the slip ring is f. In the forward-looking imaging scheme, a three-dimensional image can be obtained every time the transducer rotates by 180° around the center. When the volumetric frame rate of ultrasound four-dimensional imaging is set to f, the rotational speed of the motor and the slip ring is 0.5*f.

Optionally, the ultrasound system transmits ultrasound beams in different directions and receives the reflected echoes to form a two-dimensional image. Multiple two-dimensional images are acquired at different rotation angles around the rotation center, followed by three-dimensionally reconstruction. Reconstructed four-dimensional images are rendered and displayed in the monitor. The technology includes:
obtaining the coordinate position of each voxel on the ultrasound beam and the distance between a voxel point and each channel of the ultrasound transducer, and calculating transmit and receive delays;
calculating the space angle of each voxel point in spherical coordinates, and beamforming the two-dimensional images;
after the ultrasound transducer rotates around the central axis by 360° (a side-looking transducer) or 180° (a forward-looking transducer) each time, performing quick three-dimensional reconstruction on all the two-dimensional images in a Cartesian coordinate system according to the rotation angle;
when the volumetric frame rate of four-dimensional imaging is set to f, the rotational speed of the motor, the slip ring and the transducer is f(a side-looking transducer) or 0.5*f (a forward-looking transducer);
performing interpolation according to the rotation angle, and calculating the gray value of each rotation angle according to the coordinate information; and
performing rendering and image display of the generated four-dimensional images.

Optionally, when the transducer unidirectionally and uniformly rotates at a high speed, the transducer transmits a plurality of focusing ultrasound beams, or transmits a plurality of non-focused plane waves or diverging waves, and receives reflected echo signals. Specifically, the transducer can transmit a plurality of focusing ultrasound beams, diverging waves or plane waves in sequence for 2D imaging.

Optionally, the transducer is connected with the catheter end connector through a wire harness, the transducer wire harness being a coaxial cable or a flexible circuit board. The transducer rotates inside the sheath, and the sheath remains static.

Optionally, the transducer wire harness, the transducer and the torque coil are located in the sheath. When in use, a coupling fluid is injected into a proximal end of the catheter and air is exhausted for acoustic coupling between the ultrasound transducer and the sheath.

Specifically, an ultrasound transducer is connected to the catheter end connector electrically via coaxial cable or flexible circuit board. The received and pre-processed RF data is transferred to the ultrasound system via a PCI-E interface, or other high-speed data transmission interfaces such as USB or a network cable interface.

Optionally, the sheath is a deflectable sheath with gradually changing hardness. The catheter has a relatively soft distal end and a relatively hard proximal end. Pull wires are integrated in a catheter wall of the catheter. The catheter tip is deflected by pulling the pull wires through operation of a handle.

Specifically, a coupling fluid needs to be filled between the transducer and the sheath. Liquid may be filled in a gap between the transducer and the sheath by injecting the coupling fluid (usually normal saline) before operation and exhausting air, namely by injecting saline into the catheter. The coupling fluid will be sealed in the gap between the transducer and the sheath during surgery.

In the embodiment of the present disclosure, the motor (which may be a stepping motor, a servo motor, a linear motor, etc.), the slip ring, the host end connector and the ultrasound system are located outside the body and are used repeatedly, while the catheter (including the sheath, a torque coil, the cable, the catheter end connector and the ultrasound transducer) is disposable. During the surgery, the ultrasound catheter is inserted into the cardiac chamber by peripheral vein intervention, and the angle of a catheter tip is deflected through the handle to select a suitable imaging angle. Then, the catheter remains fixed in position, and the motor starts rotation for 4D imaging.

FIG. 1 is a schematic diagram of a forward-looking rotating scanning 4D ICE system. In the system, the motor drives the one-dimensional phase array transducer to rotate unidirectionally inside the tip of the catheter through a torque coil. In the scheme, the transducer's imaging plane is perpendicular to the catheter's axis, and the coupling fluid is filled between the transducer and the sheath for acoustic impedance matching.

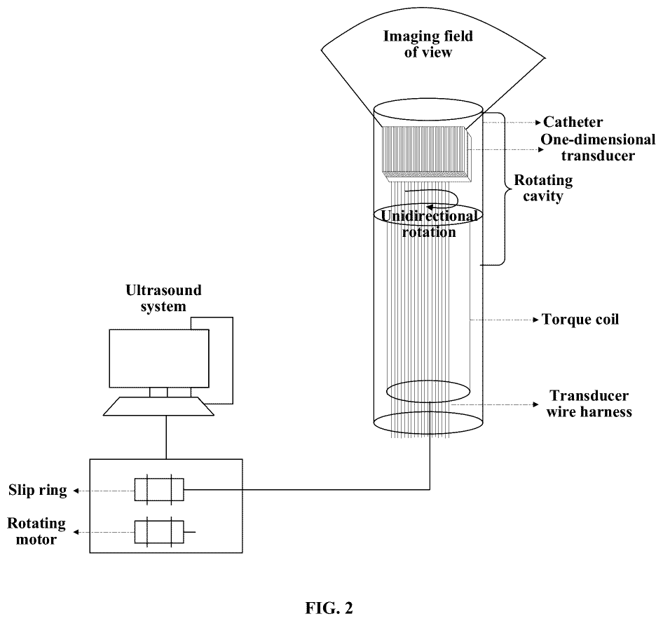

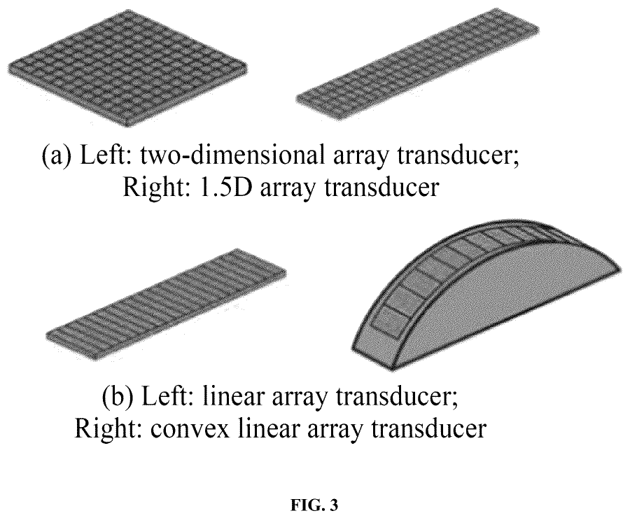

FIG. 2 is a schematic diagram of a side-looking rotating scanning 4D ICE system. The difference with FIG. 1 is that the transducer's imaging plane is parallel to the catheter's axis for side-looking imaging.

The embodiment of the present disclosure provides a forward-looking mechanical four-dimensional intracardiac ultrasound imaging system.

Firstly, the ultrasound system, the motor and the multi-channel slip ring are located outside the body. The slip ring includes a stator side and a rotor side. The motor drives the rotor of the slip ring to rotate unidirectionally and uniformly through gear drive or a synchronous belt. The stator side is static and connected to the ultrasound system. The rotor side is connected with the host end connector. A catheter end connector is connected with an ultrasonic cable and a torque coil and is single use. When in use, the catheter end connector is connected to the host end connector.

Secondly, during surgery, the motor, the rotor side of the slip ring and the host end connector rotate unidirectionally and uniformly at N rps, so that the catheter end connector, the torque coil, the cable and the ultrasound transducer rotate unidirectionally and uniformly at N rps. At the same time, the ultrasound system is electrically connected with the ultrasound transducer through the slip ring stator, the slip ring rotor, the host end connector, the catheter end connector and the cable.

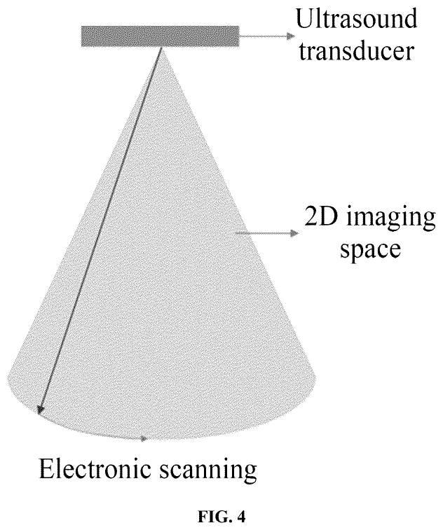

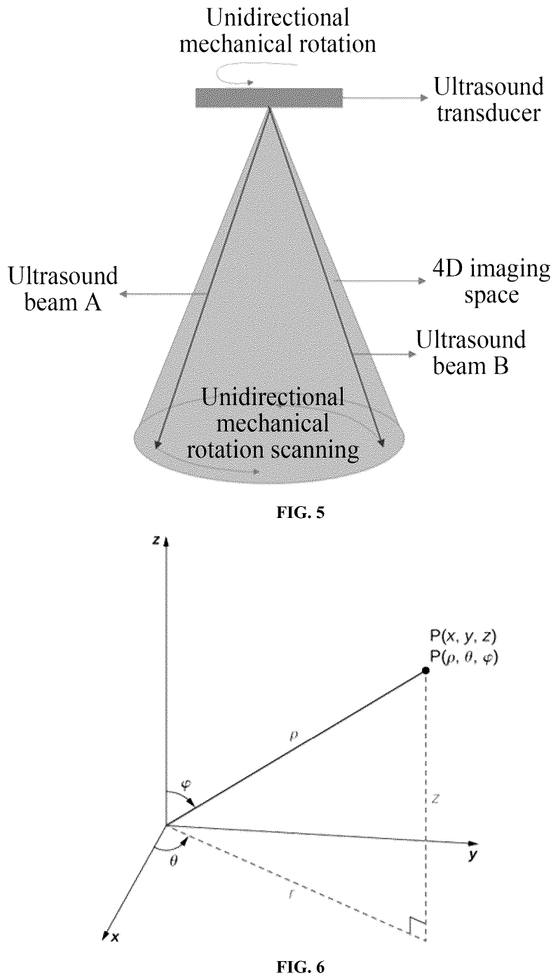

Thirdly, the transducer is a one-dimensional phase array transducer rotating around the rotation center. During imaging, the transducer rotates unidirectionally and uniformly at N rps, and performs two-dimensional ultrasound imaging while rotating. That is, ultrasound beams are transmitted and received. As shown in FIG. 5, in forward-looking imaging, ultrasound beams scan the whole space every time the transducer rotates by 180°, while for side-looking imaging the transducer rotates by 360° to scan a whole imaging space. That is, a complete three-dimensional scan of the space and a volumetric frame of 4D ICE image acquisition are completed. FIG. 4 refers to a forward-looking transducer scanning method of a linear array, that is, a two-dimensional imaging scanning method in the absence of rotation. FIG. 5 refers to a three-dimensional imaging scanning method during rotation of a forward-looking transducer.

Fourthly, when the ultrasound transducer rotates unidirectionally around the center, ultrasound beams in different directions are transmitted in sequence. The four-dimensional images can be obtained by three-dimensionally reconstructing the two-dimensional images formed at different rotation angles and rendering the images in the monitor.

Assuming that the mechanical rotation is in the azimuth direction and the ultrasound beams are scanned in the elevation direction, the reconstruction steps are as follows:
(1) assuming that there is a voxel point on each beam, the distance from the center of the transducer is denoted as p; then, each voxel is represented as (ρ,θ,φ) in spherical coordinates, wherein 0 and o are the space angles of the beam in the azimuth and elevation directions;
(2) three-dimensional reconstruction every time the transducer rotates by 180°; during the reconstruction process, there are K voxels in a four-dimensional imaging space in FIG. 4, and the spatial position of each voxel is represented as (x, y, z) in the Cartesian coordinate system, wherein;
(3) three-dimensional interpolation; according to the coordinate positions, there are various interpolation methods, including linear interpolation, polynomial interpolation and other calculation methods, and the embodiment of the present disclosure will not be described in detail;
(4) rendering and display of the generated four-dimensional images; and a three-dimensional rendering algorithm, such as a ray-casting algorithm, is performed to render the three-dimensional images on a monitor.

In the embodiment of the present disclosure, imaging is performed while the ultrasound transducer rotates around the rotation center. For the forward-looking imaging scheme, the volumetric frame rate is twice the rotational speed of the motor and the slip ring, while for the side-looking imaging scheme the volumetric frame rate is equal to the rotational speed. Therefore, the forward-looking scheme in the embodiment of the present disclosure greatly reduces the speed requirements for the motor and the slip ring.

The embodiment of the present disclosure provides a forward-looking or side-looking mechanical rotating 4D intracardiac ultrasound imaging device. Compared with existing commercial 4D ICE technology which uses a two-dimensional phase array transducer, the rotating one-dimensional phase array transducer described in the embodiment of the present disclosure can significantly reduce the cost and manufacturing difficulty of the transducer.

The embodiment of the present disclosure has the following advantages.

The embodiment of the present disclosure provides a forward-looking mechanical four-dimensional intracardiac ultrasound imaging system. The system includes an ultrasound system, a slip ring, the host end connector and the catheter end connector, a motor, a transducer, a sheath and a torque coil. The ultrasound system is connected to the transducer through the slip ring, the connectors, a cable and the torque coil in sequence. When in use, the motor drives the multi-channel slip ring to rotate unidirectionally and uniformly, thereby driving the connectors, the torque coil and the transducer to rotate unidirectionally and uniformly at a high speed in the sheath. At the same time, the transducer transmits the ultrasound beams in different directions and receives the reflected echoes to generate multiple frames of two-dimensional images rotating around the rotation center, and the two-dimensional images formed at different rotation angles are reconstructed to obtain four-dimensional images for rendering and real-time display.

The embodiments of this disclosure may use a form of a computer program product that is implemented on one or more computer-usable storage media (including but not limited to a disk memory, a CD-ROM, an optical memory, etc) that include computer usable program code.

It should be understood that these computer program instructions may be provided to a general-purpose computer, a special-purpose computer, an embedded processor, or a processor of any other programmable data processing electronic device, so that the instructions executed by a computer or a processor of any other programmable data processing electronic device comprise an apparatus for implementing a specific function in one or more processes for four-dimensional intracardiac echocardiography imaging.

These computer program instructions may be stored in a computer-readable memory that can guide the computer or any other programmable data processing electronic equipment to work in a specific manner. The instruction apparatus implements a specific function in one or more processes for the purpose of four-dimensional intracardiac echocardiography imaging.

These computer program instructions may be loaded onto a computer or another processing units, so that a series of operations are performed on the computer or another programmable device, thereby generating computer-implemented processing.

Although some preferred embodiments of the present disclosure have been described, those skilled in the art be adjusted to make changes and modifications to these embodiments without much effort. Therefore, the following claims are intended to be construed as to cover the preferred embodiments and all changes and modifications falling within the scope of the embodiment of the present disclosure.

In the description of the specification, the terms such as “one embodiment”, “some embodiments”, “examples”, “specific examples” or “some examples” indicates at least one embodiment or example of the disclosure in combination with specific characteristics, structures, materials or characteristics described by the embodiment or example. In the specification, schematic expression of the above terms does not necessarily refer to the same embodiment or example. Moreover, the described specific features, structures, materials or characteristics can be combined in other embodiments or examples appropriately. In addition, those skilled in the art can integrate and combine different embodiments or examples and features of different embodiments or examples described in this specification without contradicting each other.

Although the embodiment of the present disclosure has already been illustrated and described, it is understood that the embodiment is exemplary but cannot be understood as limiting the present disclosure, and the embodiment can be changed, amended, replaced and converted by those skilled in the art in the scope of the present disclosure.

What is claimed is:

1. A mechanical four-dimensional intracardiac ultrasound imaging system (including forward looking or side looking), comprising:
an ultrasound system, a slip ring, a host end connector, a catheter end connector, a motor, a transducer, a sheath and a torque coil, wherein the ultrasound system is connected to the host end connector through the slip ring, and the catheter end connector is connected to the transducer through the cable and the torque coil,
wherein the motor is configured to drive a rotor side of the slip ring to rotate uniformly and unidirectionally at a high speed;
wherein the slip ring comprises a rotor side and a stator side, the stator side being directly connected with the host end connector, the rotor side being connected with a catheter, and the motor driving the rotor side of the slip ring to rotate, thereby driving a rotor side connector, the torque coil and the transducer to rotate around the center of the transducer in the sheath;
wherein the torque coil is configured to drive the ultrasound transducer to rotate within the sheath;
wherein the ultrasound transducer is one-dimensional phase array, and is configured to transmit and receive ultrasound beams in different directions
wherein the ultrasound system is configured to generate two-dimensional images rotating around a central axis at a high speed according to focusing ultrasonic beams or plane waves in different directions, and wherein,
wherein the ultrasound transducer is capable of forward-looking (as shown in FIG. 2) or side-looking (as shown in FIG. 1) imaging, for side-looking imaging, the ultrasound transducer generates a three-dimensional images for a rotation of 360°, for forward-looking imaging, the ultrasound transducer generates a three-dimensional image for a rotation of 180°, as a result, when the volumetric frame rate of ultrasonic four-dimensional imaging is set to f, the rotational speed of the motor and the slip ring is 0.5*f.

2. The system according to claim 1, wherein the slip ring comprises a stator side and a rotor side, the rotor side being connected with the rotating motor and rotating, the stator side being connected to the ultrasound system and static, and the rotor side and the stator side moving relatively and being electrically connected via electric brush.

3. The system according to claim 1, wherein the transducer is a one-dimensional phase array transducer and rotates unidirectionally and uniformly at a high speed around the rotation center.

4. The system according to claim 1, wherein the catheter has a water inlet with a one-way valve in a proximal end, and an air outlet made of silica gel in a distal end, the coupling fluid being capable of being injected from the water inlet in the proximal end of the catheter, and air being exhausted through the air outlet.

5. The system according to claim 1, wherein the ultrasound system transmits and receives ultrasound beams in different directions through the transducer, and generates multiple two-dimensional ultrasound images rotating around the rotating shaft, the two-dimensional images formed at different rotation angles are rapidly three-dimensionally reconstructed, the reconstructed four-dimensional images are rendered and displayed,
obtaining the coordinate position of each voxel on the ultrasound beam and the distance from the voxel to each channel of the ultrasound transducer, and calculating transmit and receive delays;
calculating the space angle of each voxel point in spherical coordinates to reconstruct the two-dimensional images;
after the ultrasound transducer rotates around the central axis by 180° (forward looking) or 360° (side looking) each time, performing three-dimensional reconstruction on all the two-dimensional images in a Cartesian coordinate system, when the volumetric frame rate of four-dimensional imaging is set to f, the rotational speed of the motor, the slip ring and the transducer is 0.5*f for forward looking and f for side looking;
performing interpolation according to the space angle, and calculating the gray value of each space angle according to the coordinate information;
performing rendering and real-time image display of the generated four-dimensional images.

6. The system according to claim 1, wherein when the transducer unidirectionally and uniformly rotates at a high speed, the transducer transmits a plurality of focused ultrasound beams for flat scanning or non-focused plane waves for flat scanning, and receives the reflected echo signals.

7. The system according to claim 1, wherein the transducer is connected with the catheter end connector through a wire harness, the transducer wire harness being a coaxial cable or a flexible printed circuit board, the transducer rotating inside the sheath, and the sheath being deflectable and static.

8. The system according to claim 7, wherein the transducer wire harness, the transducer and the torque coil are located in the sheath, and wherein during surgery, a surgeon injects a coupling fluid into the one-way inlet near the proximal end of the catheter and the air is exhausted from the catheter to form acoustic coupling between the transducer and the sheath.

9. The system according to claim 8, wherein the sheath is deflectable with gradually changing hardness, the catheter having a relatively soft distal end and a relatively hard proximal end, and pull wires being integrated in a sheath wall of the sheath, thereby deflecting the sheath by pulling the pull wires through operating the handle.

10. The system according to claim 1, wherein the motor and the slip ring are integrated in a driver, the driver being capable of being put on a hospital bed or located on an adjustable support arm, so that the distance from the catheter to the patient can be adjusted by adjusting the position of the driver.
